SEQUENCE LISTING (24 sequences)

SEQ ID NO: 1 - protein, 1196 aa, synthetic construct
FVFLVLLPLVSSQCVNLTTRTQLPPAYTNSFTRGVYYPDKVFRSSVLHSTQDLFLPFFSNVTWFHAHVSGTNGTKRFDNPVLPFNDGVYFASTEKSNRGWFGTTLDSKTQSLLVNNATNVVKVCEFQFCNDPFLGVYYHKNNKSWMESEFRVYSSANNCTFEYVSQPFLMDLEGKQGNFKNLREFVFKNDGYFKYSKHTPNLVRDLPQGFSALEPLVDLPGNTRFQTLLALHRSYLTPGDSSSGWTAGAAAYYVGYLQPRTFLLKYNENGTTDAVDCALDPLSETKCTLKSFTVEKGYQTSNFRVQPTESVRFPNTNLCPFGEVFNATRFASVYAWNRKRSNCVADYSVLYNSASFSTFKCYGVSPTKLNDLCFTNVYADSFVRGDEVRQAPGQTGKADYNYKLPDDFTGCVAWNSNNLDSKVGGNYNYLYRLFRKSNLKPFERDSTEYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDADTTDAVRDPQTLELDTPCSFGGVSVTPGTNTSNQVAVLYQGVNCTEVPVAHADQLTPTWRVYSTGSNVFQTRAGCLGAEHVNNSYECDPGAGCASYQTQTNSPRRARSVASQSAYTMSLGAENSVAYSNNSAPTNFTSVTTELPVSMTKTSVDCTMYCGDSTECSNLLLQYGSFCTQLNRALTGAVEQDKNTQEVFAQVKQYKTPPKDFGGFNFSQLPDPSKPSKRSFEDLLFNKVTLADAGFKQYGDCLGDAARDLCAQKFNGLTVLPPLLTDEMAQYTSALLAGTTSGWTFGAGAALQPFAMQMAYRFNGGVTQNVLYENQKLANQFNSAGKQDSLSSTASALGKLQDVVNQNAQALNTLVKQLSSNFGASSVLNDLSRLDKVEAEVQDRLTGRLQSLQTYVTQQLRAAERASANLAATKMSECVLGQSKRVDFCGKGYHLMSFPQSAPHGVVFLHVTYVPAQEKNFTTAPACHDGKAHFPREGVFVSNGTHWFVTQRNFYEPQTTDNTFVSGNCDVVGVNNTVYDPLQPELDSFKEELDKYFKNHTSPDVDLGDSGNASVVNQKEDRLNEVAKNLNESLDLQELGKYEQYKWPWYWLGFAGLAVMVTMLCCMTSCCSCLKGCCSCGSCCKFDEDDSEPVLKGVKLHYT

SEQ ID NO: 2 - protein, 18 aa, synthetic construct
MDWTWILFLVAAATRVHS

SEQ ID NO: 3 - DNA, 3819 bp, synthetic construct
  source 1..3819: mol_type = other DNA
ttcgtctttctcgtgctgctgcctctggtgtccagccagtgtgtgaacctgaccaccaggacacagctgcctccagcctacaccaacagctttaccagaggcgtgtactaccccgacaaggtgttcagatccagcgtgctgcactctacccaagacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggatctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgactccttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgtctagcacagccagcgctctgggcaagctgcaggatgtggtcaaccagaatgcccaggcactgaacaccctggtcaagcagctgagcagcaatttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgcatgaccagctgctgtagctgcctgaagggctgttgtagctgtggcagctgctgcaagttcgacgaggacgattctgagcccgtgctgaaaggcgtgaagctgcactacacctaa

SEQ ID NO: 4 - DNA, 54 bp, synthetic construct
  source 1..54: mol_type = other DNA
atggactggacctggatcctgtttctggtggccgctgccacaagagtgcacagc

SEQ ID NO: 5 - DNA, 23 bp, synthetic construct
  source 1..23: mol_type = other DNA
aaggggtactgctgttatgtctt

SEQ ID NO: 6 - DNA, 23 bp, synthetic construct
  source 1..23: mol_type = other DNA
caaggtccataagaaaaggctga

SEQ ID NO: 7 - DNA, 24 bp, synthetic construct
  source 1..24: mol_type = other DNA
ttgtaatgatccatttttgggtgt

SEQ ID NO: 8 - DNA, 21 bp, synthetic construct
  source 1..21: mol_type = other DNA
ttctcttcctgttccaagcat

SEQ ID NO: 9 - DNA, 20 bp, synthetic construct
  source 1..20: mol_type = other DNA
ttgtgcccttttggtgaagt

SEQ ID NO: 10 - DNA, 21 bp, synthetic construct
  source 1..21: mol_type = other DNA
aagaacagcaacctggttaga

SEQ ID NO: 11 - DNA, 22 bp, synthetic construct
  source 1..22: mol_type = other DNA
cgtgatccacagacacttgaga

SEQ ID NO: 12 - DNA, 22 bp, synthetic construct
  source 1..22: mol_type = other DNA
tgtcttggtcatagacactggt

SEQ ID NO: 13 - DNA, 22 bp, synthetic construct
  source 1..22: mol_type = other DNA
ggctgaacatgtcaacaactca

SEQ ID NO: 14 - DNA, 21 bp, synthetic construct
  source 1..21: mol_type = other DNA
caccaaaggtccaaccagaag

SEQ ID NO: 15 - DNA, 20 bp, synthetic construct
  source 1..20: mol_type = other DNA
acacttctgcactgttagcg

SEQ ID NO: 16 - DNA, 22 bp, synthetic construct
  source 1..22: mol_type = other DNA
gccctttccacaaaaatcaact

SEQ ID NO: 17 - DNA, 22 bp, synthetic construct
  source 1..22: mol_type = other DNA
agagtgtgtacttggacaatca

SEQ ID NO: 18 - DNA, 21 bp, synthetic construct
  source 1..21: mol_type = other DNA
gcatccttgatttcaccttgc

SEQ ID NO: 19 - DNA, 22 bp, synthetic construct
  source 1..22: mol_type = other DNA
gacgtcaatgggagttttgttt

SEQ ID NO: 20 - DNA, 25 bp, synthetic construct
  source 1..25: mol_type = other DNA
atagaatgacacacctactcagaca

SEQ ID NO: 21 - DNA, 19 bp, synthetic construct
  source 1..19: mol_type = other DNA
ctgcacccagctgaataga

SEQ ID NO: 22 - DNA, 20 bp, synthetic construct
  source 1..20: mol_type = other DNA
aattgaagccgccgaagtcc

SEQ ID NO: 23 - DNA, 20 bp, synthetic construct
  source 1..20: mol_type = other DNA
gtgacgtggacatccgtaaa

SEQ ID NO: 24 - DNA, 21 bp, synthetic construct
  source 1..21: mol_type = other DNA
cagggcagtaatctccttctg

(19) United States
(12) Patent Application Publication
(10) Pub. No.: US 2026/0034209 A1
(43) Pub. Date: Feb. 5, 2026

(54) CORONAVIRUS VACCINE, PRODUCTION AND APPLICATION

(71) Applicant: TURKIYE BILIMSEL VE TEKNOLOJIK ARASTIRMA KURUMU, Ankara (TR)

(72) Inventors: MERT DOSKAYA, Izmir (TR); AYTUL GUL, Izmir (TR); SEDEF ERKUNT ALAK, Izmir (TR); HUSEYIN CAN, Izmir (TR); MUHAMMET KARAKAVUK, Izmir (TR); FATMA GULAY KORUKLUOGLU, Istanbul (TR); AYSU DEGIRMENCI DOSKAYA, Izmir (TR); HILAL YAZICI MALKOCOGLU, Kocaeli (TR); CEREN GUL, Izmir (TR); TUGBA KARAKAVUK, Izmir (TR); ERCUMENT KARASULU, Izmir (TR); ADNAN YUKSEL GURUZ, Izmir (TR); HIVDA ULBEGI POLAT, Kocaeli (TR); CEMAL UN, Izmir (TR); HASAN AKBABA, Izmir (TR); AYSE GULTEN KANTARCI, Izmir (TR); GULSAH EREL AKBABA, Izmir (TR); HUSNU PULLUKCU, Izmir (TR); MELTEM TASBAKAN, Izmir (TR); SABAN TEKIN, Istanbul (TR)

(73) Assignee: TURKIYE BILIMSEL VE TEKNOLOJIK ARASTIRMA KURUMU, Ankara (TR)

(21) Appl. No.: 19/056,786

(22) Filed: Feb. 19, 2025

(30) Foreign Application Priority Data
Jul. 31, 2024 (TR) 2024/009920

(51) Int. Cl. A61K 39/215; A61K 39/00; A61P 31/14; C07K 14/005

(52) U.S. Cl. CPC A61K 39/215; A61P 31/14; C07K 14/005; A61K 2039/53; A61K 2039/54; C12N 2770/20022; C12N 2770/20031; C12N 2770/20071

(57) ABSTRACT

An SARS-COV-2 recombinant Spike protein is provided. The research process of the protein is as follows: the dominant strain in circulation was identified by screening clinical samples of SARS-COV-2 patients and its mutations in Turkey were evaluated by sequencing the Spike gene. Sequencing data and in silico methods were used to design the Spike antigen and then the novel Spike antigen was docked with the human ACE2 (Angiotensin Converting Enzyme-2) receptor to determine the binding energy. After DNA vaccine construction, HEK293T cells were transfected and analyzed for protein expression capacity by IFA, Western blot and RT-qPCR, then BALB/c mice and K18-hACE2 transgenic mice were immunized with DNA vaccine administered intramuscularly (IM) and intradermally (ID) using an electroporator device three times on days 0, 14 and 56. Humoral and cellular immune responses were then analyzed using recombinant ELISA, Western blot, surrogate virus neutralization assay, microneutralization assay, Cytokine ELISA and flow cytometry.

### CROSS REFERENCE TO THE RELATED APPLICATIONS

This application is based upon and claims priority to Turkish Patent Application No. 2024/009920, filed on Jul. 31, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBAB109_Sequence_Listing.xml, created on Jan. 14, 2025, and is 27,001 bytes in size.

### TECHNICAL FIELD

The invention relates to the field of genetic engineering and molecular biology. The invention relates more specifically to SARS-COV-2 coronavirus Spike(S) proteins, fusion proteins containing said Spike proteins, nucleic acid molecules including nucleotide sequences encoding said Spike proteins or fusion proteins, and vectors and host cells containing these nucleic acid molecules. Furthermore, the invention relates to pharmaceutical compositions including said Spike proteins, fusion proteins, nucleic acid molecules or vectors and their use for the prevention and treatment of SARS-COV-2 coronavirus infection.

### BACKGROUND

Coronaviruses belong to a large family of RNA viruses that cause respiratory, gastrointestinal and neurological infections in humans and animals. Among the seven types of coronaviruses known to infect humans, the first four strains (HCoV-229E, HCoV-OC43, HCoV-NL63 and HCoV-HKU1) cause only mild symptoms, while the other strains have caused serious outbreaks in the 21st century; these are severe acute respiratory syndrome coronavirus (SARS-CoV), which caused an outbreak in 2003, Middle East respiratory syndrome coronavirus (MERS-CoV), which caused an outbreak in 2012, and severe acute respiratory syndrome coronavirus 2 (SARS-COV-2), which caused an outbreak in late 2019 (Chen et al. 2020; Sharma et al. 2020; Hu et al. 2021).

After SARS-COV-2 was identified as a global pandemic, millions of people died worldwide and a significant proportion of recovered patients developed post-acute sequelae of SARS-COV-2 infection (PASC or Long COVID) or multisystem inflammatory syndrome (MIS-C), which was detected in children (Zhu et al., 2020). Vaccines have reduced the consequences of COVID-19 and its complications, but studies have shown that vaccine effectiveness decreases over time (Altmann and Boyton, 2021). Although these vaccines reduced the risk of prolonged COVID, those with prolonged COVID did not benefit from vaccination (Watanabe et al., 2023). These facts point to the need for better vaccines to provide longer-lasting protection against SARS-COV-2 and variants of concern (VOC), as well as protection against COVID-19 complications such as long COVID.

DNA vaccines are among the next-generation vaccine platforms that have the potential to accelerate the production of effective vaccines when urgently needed due to their ability to elicit both humoral and cellular immune responses for prolonged periods of time, their easy production, high stability and long-term storage even at room temperature (Abbasi 2021; Silveira et al., 2021). The safety and efficacy of DNA vaccines have also been documented in many clinical studies (Momin et al., 2021; Aurisicchio et al., 2022; Kraynyak et al., 2022; Zhang et al., 2017). DNA vaccines, in particular, eliminate concerns associated with other vaccines, such as safety risks associated with replicating microorganisms or the use of any viral vector that induces anti-vector immunity and risks associated with the production of inactivated virus vaccines (Kutzler and Weiner, 2008; Silveira et al., 2021). According to the WHO list of vaccine candidates of Mar. 30, 2023, there are 17 DNA vaccine candidates in various stages of clinical development and 16 DNA vaccine candidates in various stages of preclinical development.

Chinese patent CN117448362A, included in the prior art, describes a novel recombinant nucleic acid molecule including a first DNA molecule encoding the receptor binding domain of the SARS-COV S protein and a second DNA molecule encoding the RBD domain of the S protein of the SARS-COV-2 Beta mutant strain. The document describes the utility of this molecule in the preparation of vaccines to prevent and/or treat SARS-COV and/or SARS-COV-2 virus infection.

US patent US20230364220A1, included in the prior art, describes an SARS-COV-2 spike-based DNA composition including a recombinant DNA construct in a plasmid. The DNA composition is capable of inducing the production of neutralizing antibodies when administered to humans after at least two administrations.

The Chinese patent document CN111569057A, included in the prior art, describes the COVID-19 DNA vaccine, its preparation method and application. The expression level is increased by deleting the glycosylated aspartate in the N−1 region of the SARS-COV-2 Receptor Binding Domain (RBD) sequence and the functionality and antigenicity of the DNA vaccine are protected. Meanwhile, the coding protein uses pVAX1 as vector backbone molecules and the upstream and downstream base sequences of the gene's start codon ATG are designed to follow the Kozak rule.

The international patent document WO2022105880A1, included in the prior art, does not describe the highly efficient DNA vaccine, method and use of a fusion protein for the novel coronavirus. The DNA vaccine ZD-nCor19 uses the RBD protein of the novel coronavirus, the 301-538 aa segment of the S2 subunit and the 138-369 aa segment of the N protein as target antigens. The obtained immune synergism molecule can effectively trigger both humoral immunity and cellular immunity and avoid the safety issues associated with ADE that may be generated by the full-length S protein and the full-length N protein.

Chinese patent CN112266411, included in the prior art, describes a novel coronavirus vaccine including a truncated Spike protein, a fusion protein including the truncated Spike protein, a nucleic acid molecule including a nucleotide sequence encoding the truncated Spike protein or the fusion protein, and a vector including the nucleic acid molecule and a host cell, and its application. Compared to the native Spike protein, the truncated Spike protein has a clearly enhanced expression level in cells (4-fold higher).

Within the scope of the invention described in this document, the spike gene of SARS-CoV-2 strains obtained from our country (Turkey) was sequenced and DNA vaccine was designed by bioinformatics analyses. DNA vaccines were amplified in E. coli cells and subsequently purified. Then, the immune response and protection were determined by intramuscular or electroporation administration to mice. As a result, a protective COVID-19 DNA vaccine was developed. In this invention, the Spike protein containing the D614G mutation of SARS-COV-2 is used to protect against COVID-19 infection by attaching it to a carrier plasmid that works in mammalian cells. This designed plasmid DNA vaccine is original and has not been used in any experimental study by others.

### SUMMARY

### Technical Problem the Invention Aims to Solve

The invention aims to prevent Severe Acute Respiratory Syndrome Coronavirus 2 (COVID-19) infections. DNA vaccines under clinical development mostly use the SARS-COV-2 Wuhan strain. In this study, the predominant circulating strain was identified by screening clinical samples of COVID-19 patients and a DNA vaccine specific to the mutations of SARS-COV-2 in Turkey was designed by sequencing the Spike gene.

The invention provides a nucleic acid vaccine against SARS-COV-2 optimized for expression in humans. This vaccine is based on the coding sequence of the Spike(S) protein of the strain that has become dominant due to mutations in Turkey.

In this study, clinical samples from SARS-COV-2 patients were first screened to identify the dominant circulating strain and mutations in Turkey were evaluated by sequencing the Spike gene. Sequencing data and in silico methods were used to design the Spike antigen and then the novel Spike antigen was docked with the human ACE2 (Angiotensin Converting Enzyme-2) receptor to determine the binding energy. After construction of the DNA vaccine, HEK293T cells were transfected and protein expression capacity was analyzed by IFA, Western blot and RT-qPCR. Then, BALB/c mice and K18-hACE2 transgenic mice were immunized with DNA vaccine administered intradermally (ID) using an electroporator device or intramuscularly (IM) by injection three times on days 0, 14 and 56. Humoral and cellular immune responses were then analyzed using recombinant ELISA, Western blot, surrogate virus neutralization assay, microneutralization assay, Cytokine ELISA and flow cytometry. Following challenge of K18-hACE2 transgenic mice with SARS-COV-2, macroscopic, histopathologic examination and RT-qPCR were performed and high levels of immunogenicity and protection were detected.

The present application includes a nucleic acid molecule encoding the Spike protein as described above or a fusion protein as described above.

In some applications of the invention, the nucleotide sequence encoding the Spike protein or fusion protein may be codon optimized or non-optimized according to the codon preference of the host cell (e.g., human cell).

In some applications of the invention, the nucleic acid molecule has a nucleotide sequence represented by SEQ ID NO: 3.

The invention further includes a vector including a nucleic acid molecule as described above. The term “vector” as defined in the context of this invention refers to a nucleic acid carrier vehicle in which a polynucleotide can be inserted. The vector can be transferred into a host cell by transformation, transduction or transfection, and the elements of genetic material it carries can be expressed in the host cell. Vectors can be of various types, including but not limited to plasmids, phagemids, cosmids, Yeast Artificial Chromosome (YAC), Bacterial Artificial Chromosome (BAC), bacteriophage, animal viruses, etc. Animal viruses that can be used as vectors include, but are not limited to, retroviruses, adenoviruses, adeno-associated viruses, herpes viruses, rabies viruses, baculoviruses, papilloma viruses, papova viruses. A vector may contain various elements that control gene expression, including, but not limited to, promoter sequences, transcription initiation sequences, enhancer sequences, selectable markers and reporter genes. Furthermore, the vector may also contain a replication initiation site.

As described herein, the term “host cell” may include various types of cells that may be used to transfer a vector, such as, but not limited to, prokaryotic cells, fungal cells, insect cells, or animal cells. In some applications of the invention, the animal cell may be a mammalian cell (e.g. a mouse cell, a human cell, etc.). In some applications of the invention, the mammalian cell may be a human cell (e.g., a hematopoietic cell, an epithelial cell, a liver cell, a tumor cell, a nerve cell). In some applications of the invention, the host cell may be an E. coli cell (e.g. E. coli DH5a cell); alternatively, the host cell may be a human cell (e.g. 293T cell).

The design of the expression vector can vary depending on factors such as the choice of host cell to be transformed, the desired level of expression. As described herein, proteins, fusion proteins, isolated nucleic acid molecules can be obtained by transforming a vector into a host cell. In the present application, the fusion protein is not a naturally occurring protein or a part of it. In some applications of the invention, the additional polypeptide is selected from a tag, a signal peptide or leader peptide, a detectable tag (e.g., Green Fluorescent Protein (GFP)), or any combination thereof. In some applications of the invention, the Spike proteins can be linked to a tag sequence to facilitate expression, detection and/or purification. In some applications of the invention, the Spike proteins can be linked to a signal peptide or leader sequence to direct release. In some applications of the invention, the signal peptide is selected from the natural signal peptide (SEQ ID NO: 2) of IgE (immunoglobulin E).

Signal peptides are not limited to those listed above, but the use of a suitable signal peptide for a desired purpose is also within the scope of the invention.

Furthermore, the present application provides a method of expressing or producing a Spike protein as described above or a fusion protein as described above. The method includes the use of a nucleic acid molecule as specified in the scope of the invention or a vector as specified in the scope of the invention or a host cell as specified in the scope of the invention.

In certain applications, the method includes expressing and/or purifying a nucleic acid molecule or a vector specified in the scope of the invention in a host cell under conditions permitting expression of the protein.

The present application further includes the use of a nucleic acid molecule or a vector as specified in the invention or a host cell to express or produce a Spike protein or a fusion protein. In certain preferred applications, the nucleic acid molecule or vector may be used for the expression or production of the Spike protein or fusion protein in-vivo. In certain preferred applications, the nucleic acid molecule or vector may be used for expression or production of Spike protein or fusion protein in a patient (e.g., a mammal, such as a human).

The present application may be used as a pharmaceutical composition including the Spike protein or fusion protein specified in the invention or a nucleic acid molecule or a vector specified in the invention and optionally combined with a pharmaceutically acceptable carrier and/or excipient.

In certain applications, the pharmaceutical composition is administered by electroporation or injection.

In certain applications, an effective amount (e.g., an effective amount for preventing or treating a novel coronavirus infection) includes a Spike protein or fusion protein or nucleic acid molecule or vector.

In certain applications, the pharmaceutical composition may be in unit dose form.

In certain applications, the pharmaceutical composition is a vaccine.

In certain applications, the pharmaceutical composition may include an adjuvant.

In certain applications, the pharmaceutical composition is a protein vaccine including a Spike protein as described above or a fusion protein as described above.

In certain applications, the pharmaceutical composition is a nucleic acid vaccine including a nucleic acid molecule as described above or a vector as described above.

In certain applications, the nucleic acid vaccine is a DNA vaccine.

### BRIEF DESCRIPTION OF THE DRAWINGS

FIGS. 1A-1J. Construction of pcoSpikeD614G DNA vaccine and in vitro expression in HEK293T cells.

FIG. 1A: Demonstration of pcoSpikeD614G plasmid design at 6785 bp size by SnapGene.

FIG. 1B: Agarose gel image showing double cutting of the pcoSpikeD614G plasmid with Nhe1 and XbaI restriction enzymes. Lane 1: DNA ladder; Lane 2: Circular pcoSpikeD614G (red arrowhead); Lane 3: circular empty pVAX1 vector; Lane 4: double-cut pcoSpikeD614G plasmid, the upper red arrowhead represents the famous spliced coSpikeD614G gene with IGHE signal peptide (3878 bp) and the lower red arrowhead represents the linearized pVAX1 plasmid (2907 bp). The original pVAX1 plasmid was 2999 bp in size and reduced to 2907 bp after removal of the multiple cloning site with the restriction enzymes Nhe1 and XbaI.

FIGS. 1C-1H: IFA images showing in vitro transfection of HEK293T cells with pcoSpikeD614G and empty pVAX1 plasmid. Scale bars represent 50 μm. C and F show DAPI-stained nuclei of cells transfected with empty pVAX1 plasmid and pcoSpikeD614G. As shown in D/E and G/H, coSpikeD614G protein expression is absence in cells transfected with pVAX1 but abundantly expressed in cells transfected with pcoSpikeD614G.

FIG. 1l: Western blot image showing the presence of coSpikeD614G protein expression in HEK293T cell lysates. Lane 1: Protein ladder; Lanes 2 and 3: lysate of cells transfected with pcoSpikeD614G was probed with pooled vaccinated mouse serum immunized three times by ID+EP and IM routes. Red arrowheads indicate recombinant pcoSpikeD614G proteins with size above about 143 kDa due to glycosylation; Lane 5: lysate of cells transfected with empty pVAX1 immunized with empty pVAX1, probed with pooled inoculated mouse serum. Lanes 4 and 6 represent pcoSpikeD614G and empty pVAX1 transfected cell lysate probed with anti p-actin antibody. Blue arrowheads indicate β-actin expression with a mw of about 42 kDa.

FIG. 1J: RT-qPCR results show significant coSpikeD614G transgene expression.

FIGS. 2A-2G. Animal studies and the humoral immune response elicited by the pcoSpikeD614G DNA vaccine.

FIG. 2A: Short timeline of animal studies. BALB/c mice were vaccinated to assess immunogenicity and K18-hACE2 transgenic mice were vaccinated to determine the protective efficacy conferred by the pcoSpikeD614G DNA vaccine.

FIG. 2B: Western blot image shows the presence of anti-S1 and anti S1+S2 antibody responses in the sera of vaccinated BALB/c mice. Lane 1: Protein ladder; Lanes 2 and 3: Recombinant S1 protein probed with pooled pcoSpikeD614G vaccinated sera from mice vaccinated three times by ID+EP and IM routes. Red arrowheads indicate recombinant S1 proteins with a size above about 76.41 kDa due to glycosylation; Lanes 4 and 5: Recombinant S1+S2 protein pooled with sera from pcoSpikeD614G-vaccinated mice vaccinated three times via ID+EP and IM routes. Blue arrowheads indicate recombinant S1+S2 proteins with a size above about 138. 5 kDa due to glycosylation; Lanes 6-7 and 8-9: recombinant S1 and S1+S2 proteins probed with pooled vaccinated mouse sera treated with empty pVAX1.

FIG. 2C: S1 IgG kinetics from mice immunized with pcoSpikeD614G administered via anti-ID+EP and IM routes showed a significant increase at day 70 compared to controls.

FIG. 2D: IgG2a/IgG1 polarization was assessed for empty pVAX1 and pcoSpikeD614G at days 0 and 70 and IgG2a responses were significantly higher.

FIG. 2E: The IgG2a/IgG1 ratio was slightly higher for pcoSpikeD614G administered via IM.

FIG. 2F: SARS-COV-2 Surrogate Virus Neutralization Assay showed that the inhibition potential of diluted sera from mice vaccinated at day 70 was significantly higher than controls.

FIG. 2G: SARS-COV-2 50% neutralization titers (VNT50) in the sera of mice vaccinated with pcoSpikeD614G, calculated at day 70. Data are presented as GMT±SD.

FIGS. 3A-3D. Cellular immune response elicited by the pcoSpikeD614G DNA vaccine.

FIG. 3A: Cytokine levels calculated by ELISA from supernatants of cultured splenocytes stimulated with the peptide pool. The IFN-γ response elicited by pcoSpikeD614G administered by ID+EP and IM routes was increased 3.15 and 2.1-fold, respectively.

FIG. 3B: Ratio of IFN-γ-secreting CD8+ cells measured by flow cytometry from cultured splenocytes stimulated with the peptide pool. The highest increase in the ratio of IFN-γ secreting CD8+ cells was obtained in mice immunized with pcoSpikeD614G administered by ID+EP. Results represent the ratio of IFN-γ secreting CD8+ cells to total CD8+ cells.

FIG. 3C: Ratio of IFN-γ-secreting CD4+ cells measured by flow cytometry from cultured splenocytes stimulated with the peptide pool. The highest increase in the ratio of IFN-γ secreting CD4+ cells was obtained in mice vaccinated with pcoSpikeD614G administered by ID+EP. Results represent the ratio of IFN-γ secreting CD4+ cells to total CD4+ cells.

FIG. 3D: Ratio of IL-4-secreting CD4+ cells measured by flow cytometry from cultured splenocytes stimulated with peptide pool. The highest increase in the ratio of IL-4 secreting CD4+ cells was obtained in mice immunized with pcoSpikeD614G administered by ID+EP. Results represent the ratio of IL-4 secreting CD4+ cells to total CD4+ cells.

FIGS. 4A-4J. Protection conferred by pcoSpikeD614G DNA vaccine in K18-hACE2 transgenic mice challenged with SARS-COV-2.

FIG. 4A: Gross pathologic scoring of the lungs to assess the level of pneumonia. Healthy lungs were scored with 0, edema-hyperemia with 0.5-1, pneumonia lesions with 1.5-5 and dead mice with 5.

FIG. 4B: Mean Ct values of RT-qPCR targeting the NC gene of SARS-COV-2 obtained from the lungs of mice. Virus load is represented by cycle threshold (Ct) values. Low Ct values represent low virus load.

FIG. 4C: Histopathology scoring of the lungs to assess inflammation status. Absence of inflammation was indicated by 0 and various levels of inflammation were scored from 1-3. Three lungs from the control group and one lung from the group of mice immunized with pcoSpikeD614G administered by IM were not scored.

FIGS. 4D-4E: In the group of mice immunized with pcoSpikeD614G administered by IM, images show normal lungs at 4× and 10× magnifications. Arrow indicates alveoli with normal morphology

FIGS. 4F-4G: In mice immunized with pcoSpikeD614G administered by ID+EP, images show normal lungs at 4× and 10× magnifications. Arrow indicates alveoli with normal morphology.

FIGS. 4H-4I: In control mice, images show normal lungs at 4× and 10× magnifications. Asterisks indicate areas of lymphocyte infiltration in the interalveolar spaces.

FIG. 4J: Kaplan-Meier Survival analysis after intranasal instillation of 105 TCID50 viruses for 3 consecutive days.

### DETAILED DESCRIPTION OF THE EMBODIMENTS

### Identification of Circulating SARS-COV-2 Variant for Vaccine Antigen Design

Before designing the Spike antigen to be used in the DNA vaccine, the Spike gene of SARS-COV-2 strains (n=20) isolated from hospitalized patients in seven different provinces of Turkey (Ankara, Adana, Antalya, Istanbul, Izmir, Trabzon, Erzurum and Van provinces) in June 2020 was sequenced. RNA samples from these patients were provided by the National Virology Reference Center Laboratory (General Directorate of Public Health, Ministry of Health, Turkey) and cDNA was synthesized using the Superscript III First-Strand Synthesis System kit (Thermo Fisher Scientific, USA) according to the manufacturer's instructions. For sequencing, the Spike gene of the Wuhan isolate (Genbank Accession no: NC_045512.2) was divided into seven fragments and each fragment was amplified with different primer pairs (Table 1). According to the results, three different mutations were detected: C882T (n=1), G906T (n=1) and A1841G (n=17). Among these mutations, only the A1841G mutation caused a D614G change in the amino acid sequence of 17 isolates. These mutations were detected in patients from Istanbul (n=3), Izmir (n=3), Antalya (n=3), Adana (n=3), Trabzon (n=3), Ankara (n=2), Erzurum (n=2) and Van (n=1).

TABLE 1
Sequence primers designed with the primer
design tool for the Spike gene of SARS-
COV-2 (NCBI; https://www.ncbi.nlm.nih.gov/
tools/primer-blast/)

| | Product |
|---|---|
| | size |
| Primer pairs | (bp) |
| F1: | 681 |
| 5-AAGGGGTACTGCTGTTATGTCTT-3 | |
| (SEQ ID NO: 5) | |
| R1: | |
| 5-CAAGGTCCATAAGAAAAGGCTGA-3 | |
| (SEQ ID NO: 6) | |
| F2: | 668 |
| 5-TTGTAATGATCCATTTTTGGGTGT-3 | |
| (SEQ ID NO: 7) | |
| R2: | |
| 5-TTCTCTTCCTGTTCCAAGCAT-3 | |
| (SEQ ID NO: 8) | |
| F3: | 831 |
| 5-TTGTGCCCTTTTGGTGAAGT-3 | |
| (SEQ ID NO: 9) | |
| R3: | |
| 5-AAGAACAGCAACCTGGTTAGA-3 | |
| (SEQ ID NO: 10) | |
| F4: | 474 |
| 5-CGTGATCCACAGACACTTGAGA-3 | |
| (SEQ ID NO: 11) | |
| R4: | |
| 5-TGTCTTGGTCATAGACACTGGT-3 | |
| (SEQ ID NO: 12) | |
| F5: | 713 |
| 5-GGCTGAACATGTCAACAACTCA-3 | |
| (SEQ ID NO: 13) | |
| R5: | |
| 5-CACCAAAGGTCCAACCAGAAG-3 | |
| (SEQ ID NO: 14) | |
| F6: | 521 |
| 5-ACACTTCTGCACTGTTAGCG-3 | |
| (SEQ ID NO: 15) | |
| R6: | |
| 5-GCCCTTTCCACAAAAATCAACT-3 | |
| (SEQ ID NO: 16) | |
| F7: | 809 |
| 5-AGAGTGTGTACTTGGACAATCA-3 | |
| (SEQ ID NO: 17) | |
| R7: | |
| 5-GCATCCTTGATTTCACCTTGC-3 | |
| (SEQ ID NO: 18 | |

### In Silico Modeling of Spike Protein and Vaccine Antigen Design

Since 85% (17/20) of the isolates contained the A1841G mutation, the Spike protein containing the D614G variation was used as the vaccine antigen (named coSpikeD614G). 3D structural models of the SARS-COV-2 Spike protein were generated by I-TASSER Server (http://zhanglab.ccmb.med.umich.edu/I-TASSER) (Yang et al., 2015) and the resulting models were refined with 3Drefine (http://sysbio.rnet.missouri.edu/3Drefine/) using RWplus model analysis (Bhattacharya et al., 2016). The refined models were evaluated with the ProSA-web tool (https://prosa.services.came.sbg.ac.at/prosa.php) for structural validation analysis (Sippl, 1993; Wiederstein and Sippl, 2007). The refined and validated 3D models were visualized and compared in the UCSF Chimera1. 14 tool (Pettersen et al., 2004). All docking analyses of the spike protein models with Native Human Angiotensin Converting Enzyme-Related Carboxypeptidase (ACE2) (RCSB PDB ID no: 1R42) were performed by ClusPro Server (https://cluspro.bu.edu/home.php) (Kozakov et al, 2017) and visualized in the UCSF Chimera 1.14 tool. coSpikeD614G and the Spike protein of the Wuhan isolate docked with the human ACE2 receptor and the docking results showed a lower energy score (−1014. 1 energy score) between coSpikeD614G and the human ACE2 receptor compared to the Spike protein of the Wuhan isolate (−950.4 energy score), indicating a stronger binding affinity between the coSpikeD614G protein and the human ACE2 receptor.

### Development of a DNA Vaccine

After analysis of the sequence data, the gene encoding the Spike protein containing the D614G mutation was codon optimized for expression in Homo sapiens (designated coSpikeD614G) and synthetically produced in the pEX-A258/coSpikeD614G vector (Eurofins, Luxembourg) in a application of the invention.

In a preferred application of the invention;

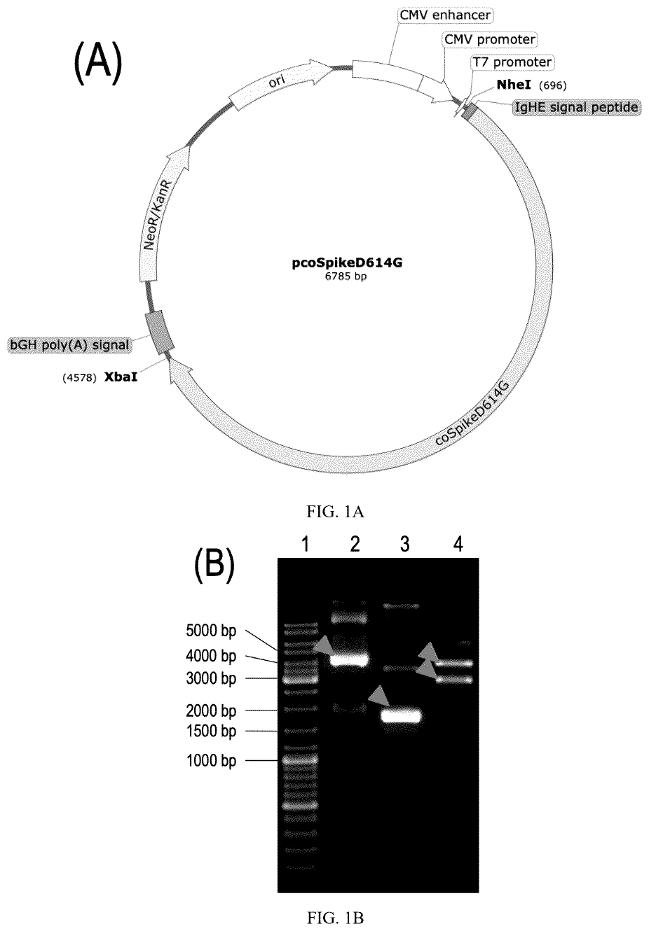

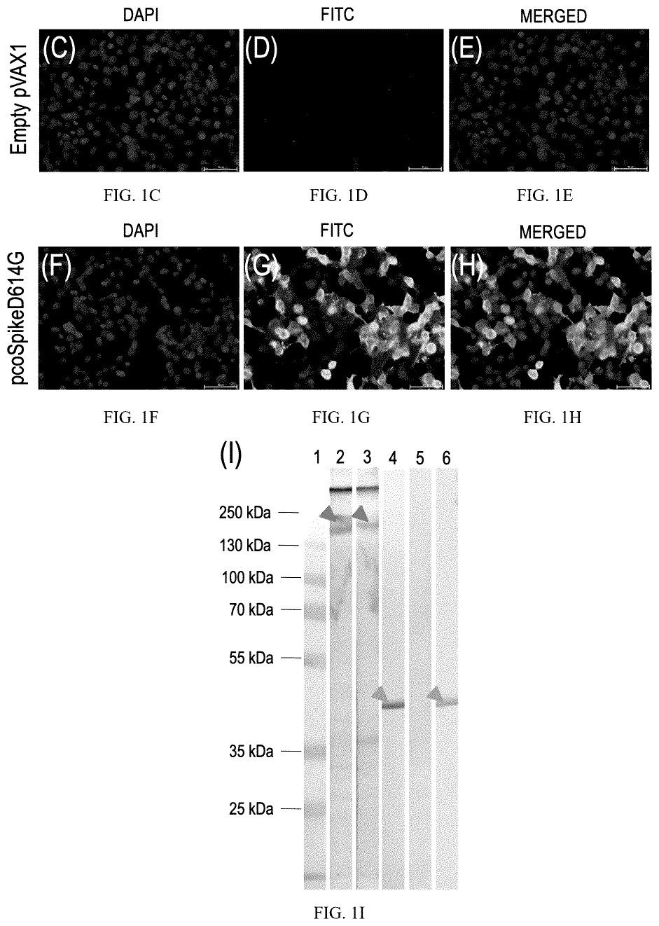

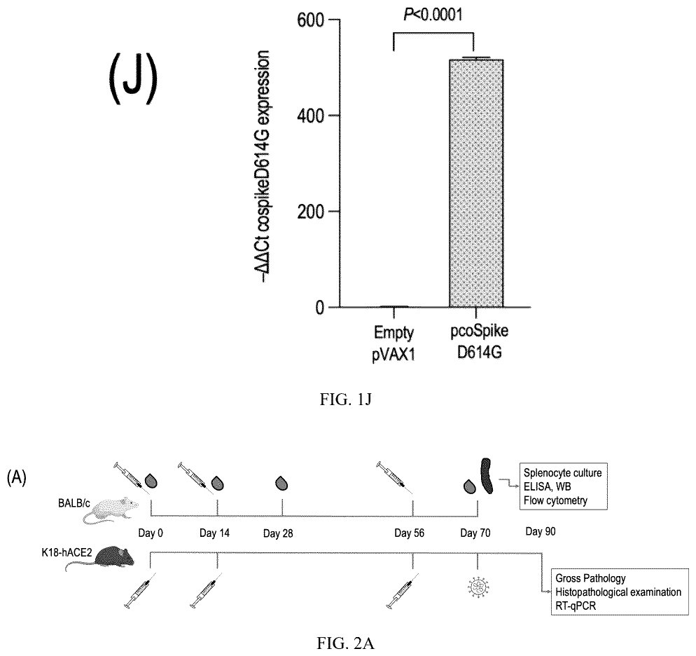

A signal peptide of the Homo sapiens Ig heavy chain epsilon-1 (V-D-J region) (IGHE) gene (GenBank accession no: AH005278.2; between 4-54 bp; fragment size 51 bp) was incorporated into the 5′-end of coSpikeD614G in frame (SEQ. ID NO X) (Table 2) to release the Spike protein outside the host cell. Furthermore, to enable cloning into the linearized and dephosphorylated pVAX1 vector (ThermoFisher Scientific, USA), a Kozak sequence (5′-end in frame) and restriction enzyme sites NheI (5′-end) and XbaI (3′-end) were included in the construct. After cloning coSpikeD614G into the pVAX1 vector, the resulting DNA vaccine plasmid was named pcoSpikeD614G, which expresses the Spike protein with a theoretical MW of 143.1 kDa (FIG. 1A).

Plasmids were transformed into chemically competent E. coli DH5a host cells (ThermoFisher Scientific, USA) and grown overnight in LB medium supplemented with kanamycin. Positive colonies were subjected to double digestion [using NheI (NEB, USA) and XbaI (NEB, USA)] and confirmed by sequencing (Forward primer: 5-GACGTCAATGGGAGTTTTGTTT-3 (SEQ ID NO: 19) and reverse primer: 5-ATAGAATGACACACCTACTCAGACA-3 (SEQ ID NO: 20)). Master cell bank and working cell bank samples prepared from glycerol stocks of overnight culture were stored in −80° C. freezer. DNA vaccine and control (empty plasmid) were produced by inoculating glycerol stocks into kanamycin-supplemented LB medium and purified using the Purelink HiPure Expi Plasmid Megaprep Kit according to the manufacturer's protocol (ThermoFisher Scientific, USA) (FIG. 1B).

In Vitro Transfection of DNA Vaccine into HEK293T Cells

In a preferred application of the invention;

To evaluate the protein expression level of pcoSpikeD614G, human embryonic kidney cells (HEK293T, ATCC CRL-3216) were cultured in six-well plates (Nunc, USA) and 4-well slides (Nunc, USA) at an initial density of 1×10⁶ and 2×10⁵ cells per well, respectively (Gül et al., 2022). HEK293T cells were transfected with 2.5 μg pcoSpikeD614G/well for six-well plates and 0.5 μg pcoSpikeD614G/well for 4-well slides using Lipofectamine 3000 (ThermoFisher Scientific, USA) reagents according to the manufacturer's instructions. 48 hours after transfection, cells were harvested from the 6-well plate, lysed with RIPA buffer (ThermoFisher Scientific, USA) and the presence of expressed proteins was determined by Western blotting. Western blot results confirmed the expression of recombinant coSpikeD614G protein with a molecular weight above about 143 kDa due to glycosylation (FIG. 1I). In the second set of 6-well plates, the mRNA expression levels of coSpike614G protein were assessed by RT-qPCR. In addition, 4-well slides were used in IFAT to demonstrate the presence of expressed Spike protein. pVAX1 without insert was used as a negative control.

### IFAT

In a preferred application of the invention;

To demonstrate the presence of coSpikeD614G protein expression, transfected HEK293T cells in 4-well slides were fixed with methanol for 5 minutes followed by cold acetone for 30 seconds. The slides were then washed with 1×PBS for 5 min, permeabilized with 0.1% Triton-X for 15 min, washed again with 1×PBS for 5 min and blocked with 1% BSA in 1×PBS for 30 min at room temperature (RT). Next, cells were probed with mouse serum (obtained from mice inoculated with pcoSpikeD614G) at 1:50 dilution for 1 hr at RT, washed 3 times for 5 min and then stained with anti-mouse IgG antibody conjugated with FITC (Sigma Aldrich, USA) at 1:100 dilution for 1 hr at RT. Next, the slides were washed with 1×PBS for 5 min, mounted with DAPI Fluoromount-G (SouthernBiotech, USA) overnight and protein expression was visualized using a fluorescence microscope (Nikon, Japan) (FIGS. 1C-1H).

### RT-qPCR

In a preferred application of the invention;

To determine the mRNA level of the Spike gene expressed by pcoSpikeD614G, total RNA was extracted from transfected HEK293T cells in 6-well plates using the RNeasy Mini Kit (Qiagen, USA) according to the manufacturer's instructions. RNA concentrations were determined by Nanodrop (ND-1000, ThermoFisher Scientific, USA). Reverse transcription reaction was performed with the SuperScript™ III First-Strand Synthesis System (ThermoFisher Scientific, USA) using 100 ng of each RNA sample and oligoDT primer provided by the kit according to the manufacturer's protocol. RT-qPCR amplification was performed using cDNA and specific codon optimized Spike gene primers (Sybr forward 5-CTGCACCCAGCTGAATAGA-3 (SEQ ID NO: 21); Sybr reverse 5-AATTGAAGCCGCCGAAGTCC-3 (SEQ ID NO: 22)) and β-actin gene primers (β-actin forward 5-GTGACGTGGACATCCGTAAA-3 (SEQ ID NO: 23); β-actin reverse 5-CAGGGCAGTAATCTCCTTCTG-3 (SEQ ID NO: 24)). The PCR reaction contained 1 μl of cDNA, 1 μM of each primer and 5 μl of LightCycler® 480 SYBR Green I Master (Roche, Germany) and was performed with a 1.5 LightCycler Real Time instrument (Roche, Germany) at 95° C. for 5 minutes followed by 10 seconds at 95° C., 20 seconds at 60° C. for β-actin primers or 40 cycles at 58° C. and 30 seconds at 72° C. for spike primers. In addition to the negative transfection control (RNA sample from cells transfected with empty pVAX1), NTC containing distilled water was included as control. LightCycler software, Version 3.5 (Roche, Germany) was used to generate the threshold cycle (CT) of each sample and 2-ΔΔCt analysis was performed to analyze the mRNA expression level of the pcoSpikeD614G plasmid. A significant level of coSpikeD614G transgene expression was confirmed by RT-qPCR compared to the control transfected with the empty pVAX1 plasmid (P<0.0001) (FIG. 1J).

### Immunization

In a preferred application of the invention;

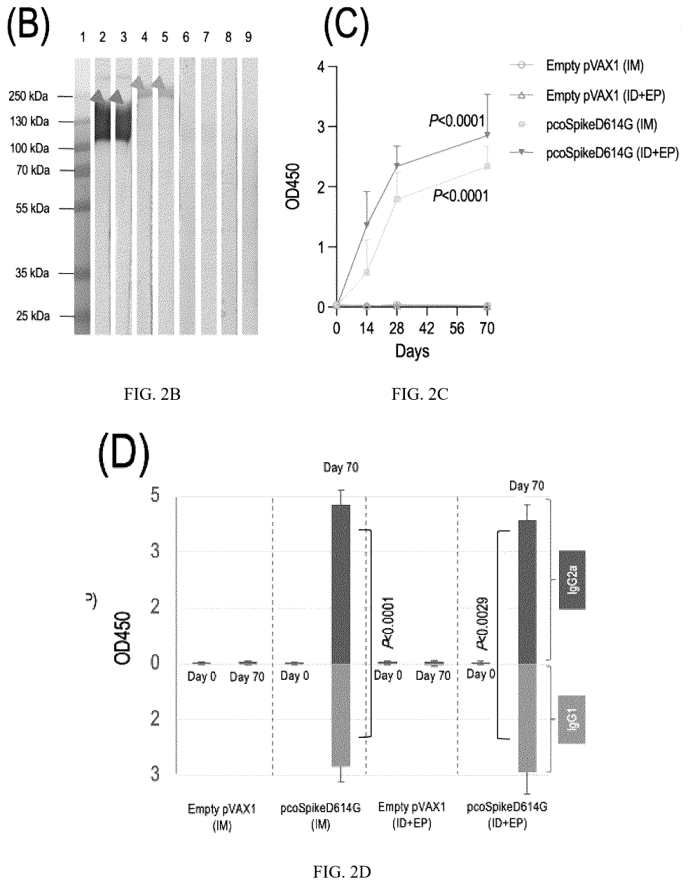

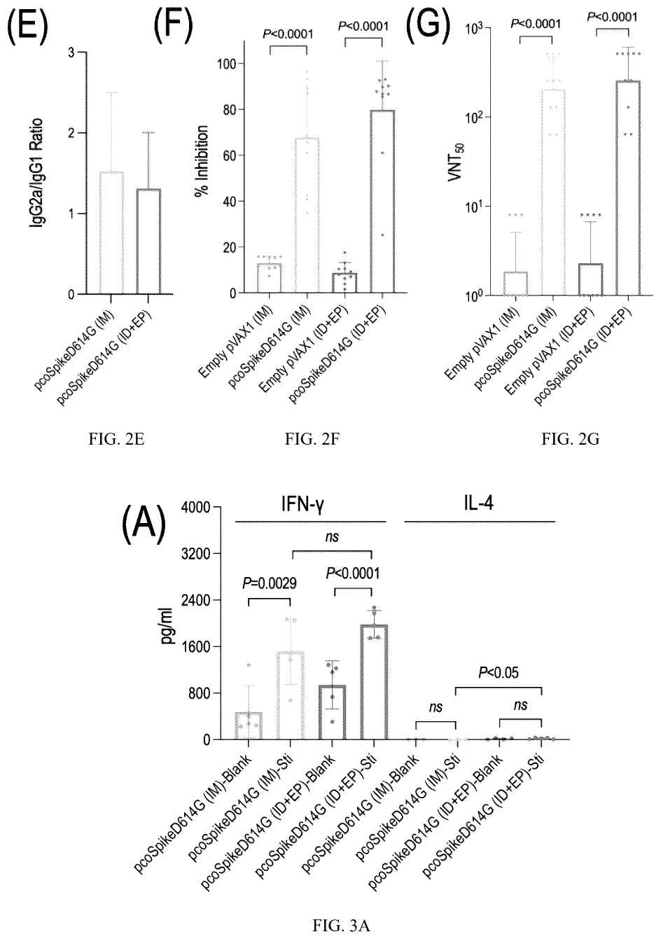

The immunogenicity study of the pcoSpikeD614G vaccine was performed in 6-8 week old female BALB/c mice purchased from the Guinea Pig Experimental Animals Laboratory (Ankara, Turkey), while 8-10 week old female K18-hACE2 transgenic mice provided by TÜBİTAK MAM were used to determine the protective efficacy of the pcoSpikeD614G vaccine. BALB/c mice (15 mice/group) and K18-ACE2 transgenic mice (10 mice/group) were treated with 1× Dulbecec three times on days 0, 14 and 56. days 0, 14 and 56 were inoculated three times under anesthesia with pcoSpikeD614G diluted in 1× Dulbecco's phosphate buffered saline (ThermoFisher Scientific USA) [ketamine hydrochloride (Ketalar) 2 mg/kg (1 ml-5 mg)+2% xylazine (Alfazyne) 3 mg/kg (1 ml-20 mg) intraperitoneally]. The pcoSpikeD614G vaccine or negative control containing the empty pVAX1 plasmid was administered intradermally (ID; 25 μg plasmid/dose) into the lumbar dermis with a 12.7 mm×30 gauge needle using an electroporation (EP) device (AgilePulse, USA) or intramuscularly (IM; 100 μg plasmid/dose) into the anterior tibial muscle with a 26 gauge needle (FIG. 2A). Before and two weeks after each inoculation, blood samples were collected by tail bleeding under anesthesia and sera were separated by centrifugation at 3000 rpm for 10 minutes and stored at −20° C. until used.

### SDS-PAGE and Western Blot

In a preferred application of the invention;

Recombinant S1 and S1+S2 proteins (SinoBiological, China) and/or Spike protein produced in transfected HEK293T cells were used in SDS-PAGE and Western Blot assay. Proteins were separated by SDS-PAGE and transferred to PVDF membrane (Immobilon-P, Millipore, USA) as described (Döşkaya et al., 2007). Membranes were incubated with pools of immunized mouse serum as primary antibodies diluted 1:50 or with anti-β actin (diluted 1:3000, Sigma-Aldrich, USA) or anti poly-His (1:3000, Sigma Aldrich, USA) antibodies for 1.5 h at RT. The membranes were then probed with alkaline phosphatase-conjugated goat anti-mouse IgG antibody (Sigma Aldrich, USA) diluted to 1:2000 for 1 hour at RT. The stains were then visualized with alkaline phosphatase-enhancing buffer (0.1 M Na₂CO₃, pH 9.5, 0.1 M NaCl, 5 mM MgCl₂) mixed with 5-bromo-4-chloro-3-indolyl phosphate (BCIP) and nitro blue tetrazolium (NBT) (Applichem, Germany). Western blot analysis of sera of pcoSpikeD614G vaccinated mice diluted 1:50 and collected on day 70 (two weeks after the third vaccination) showed strong anti-S1 and anti-S1+S2 IgG antibody responses (FIG. 2B).

### Recombinant ELISA

In a preferred application of the invention; To measure and evaluate the kinetics of the anti-S1 IgG response as well as to determine IgG2a/IgG1 polarization, recombinant ELISA was performed with 1:100 diluted mouse sera collected on day 0 and two weeks after each vaccination (days 14, 28 and 70) (Döşkaya et al., 2014; Gedik et al., 2016).

Briefly, microtiter plates (Nunc, USA) were coated overnight at 4° C. with 0.5 μg/well recombinant S1 protein (Sino Biological, China) containing the D614G mutation diluted in 1×PBS. The next day, plates were washed three times with 1×PBS-T and blocked with 0.5% non-fat dry milk in 1×PBS-T for 30 min at 37° C. Then, the plates were washed three times and probed with 100 μl mouse serum diluted 1:100 in blocking buffer for 2 hours at RT. The plates were then washed three times and probed with peroxidase-conjugated anti-mouse IgG (Sigma, USA), IgG1 (Santa Cruz, USA) and IgG2a (Santa Cruz, USA) antibodies diluted 1:3000, 1:2500 and 1:1500, respectively, in 1×PBS-T for 1 hour at RT. Then 100 μl of tetramethylbenzidine substrate solution (ThermoFisher Scientific, USA) was added to each well and the reaction was stopped with IN H2SO4. The results were analyzed on a microplate reader (Bio-Tek EL×808, USA) at 450 nm. ELISA results showed that pcoSpikeD614G administered by ID+EP or IM routes induced significantly higher anti-S1 IgG responses and increased after each inoculation compared to controls (P<0.0001). Mice vaccinated with empty pVAX1 did not induce any anti-S1 IgG responses by either route (FIG. 2C). IgG2a/IgG1 polarization assays showed that pcoSpikeD614G administered by ID+EP (P=0.0029) or IM (P<0.0001) routes induced significant IgG2a responses compared to IgG1 responses at day 70, indicating a Th1-biased immune response with each vaccination (FIG. 2D). In addition, the IgG2a/IgG1 ratio at day 70 was slightly higher in the pcoSpikeD614G group mice administered by the IM route compared to the ID+EP route (FIG. 2E).

### SARS-COV-2 Surrogate Virus Neutralization Test

In a preferred application of the invention; Neutralizing antibody responses in vaccinated mouse sera were analyzed using the ELISA-based SARS-COV-2 surrogate Virus Neutralization Test (sVNT) (AffinityImmuno, Canada), an S1 protein/ACE2 ligand binding assay according to the manufacturer's protocol. First, mouse sera diluted 1:10 and calibrators provided by the kit were added to the pre-coated wells of the plate. Then, detection reagent was added to each well and the plate was incubated for 1 hour at RT. After washing and adding tetramethylbenzidine substrate solution to each well, the reaction was stopped with IN H₂SO₄ and the results were evaluated at 450 nm versus 620 nm on a microplate reader (Bio-Tek EL×808, USA). Neutralizing antibody levels were calculated from a standard curve generated by plotting the absorbance values obtained from each standard. The results of the SARS-COV-2 Surrogate Virus Neutralization Assay showed that even 1:10 diluted sera collected on day 70 from mice administered pcoSpikeD614G via ID+EP and IM routes had significantly higher inhibition rates compared to controls (P<0.0001) (FIG. 2F).

### Microneutralization Test (MNT)

In a preferred application of the invention; Vero E6 cells from the American Type Culture Collection (ATCC: CRL-1586) were maintained in an incubator at 37° C. under 5% CO₂ using Dulbecco's Modified Eagle's Medium (DMEM) supplemented with 10% fetal bovine serum and 1% penicillin/streptomycin (ThermoFisher Scientific, USA) (Perera et al., 2020).

SARS-COV-2 virus detected from a combined nasal and throat swab of a COVID-19 patient was grown in Vero E6 cells in the BSL-3 facility at the National Virology Reference Center Laboratory, Ankara, Turkey. The virus was designated hCoV-19/Turkey/27/2020 and passaged twice in Vero E6 cells. Stock virus was harvested, aliquoted and stored at −80° C. until used.

Microneutralization testing (MNT) was performed at the BSL-3 facility located at the National Virology Reference Central Laboratory, Ankara, Turkey. Virus stock was titrated at serial log 10 dilutions (dilution factor was 10) on Vero E6 cells in a microtiter plate to obtain a 50% tissue culture infectious dose (TCID50). Plates were incubated at 37° C. under 5% CO₂ for 4 days and observed daily for cytopathic effect (CPE). The endpoint of viral dilution leading to CPE in 50% of inoculated wells (TCID50) was calculated by the Reed-Muench method (Reed and Muench, 1938).

Heat inactivated vaccinated mouse sera were serially diluted 2-fold in a microtiter plate starting at 1:4 in DMEM supplemented with 2% FBS. Then, an equal volume of 100 TCID 50 of SARS-COV-2 produced as described above was added to the serum dilutions and incubated for 1 h at 37° C. under 5% CO₂. Then, 100 μl of Vero E6 cells (2×10⁵ cells/ml in DMEM supplemented with 2% FBS) were added to the virus+serum mixture and plates were incubated at 37° C. under 5% CO₂ for 4 days.

Virus dilution was back titrated by replacing serum with medium in each experiment to determine the virus test dose. Neutralization was assessed by CPE using phase contrast microscopy. Complete inhibition of virus spread in a single well was considered a positive result. The neutralization endpoint titer was determined as the highest serum dilution that inhibited virus infection in 50% of the inoculated wells (Virus Neutralization Titer 50-VNT50) (WHO, 2013). As a result of the microneutralization assay, the geometric mean titers (GMTs) of the VNT50 values obtained from the sera of pcoSpikeD614G administered by ID+EP and IM routes were 207.9 and 256, respectively, which were significantly higher than the controls (P<0.0001) (FIG. 2G).

### Splenocyte Isolation and Stimulation

In a preferred application of the invention; to determine the cellular immune response, the spleens of mice (5 mice/group) were aseptically removed two weeks after the third vaccination. Single cell suspensions were prepared as previously described (Döşkaya et al., 2007). A total of 5×10⁵ live splenocytes were stimulated ex vivo with the previously described peptide pool (Smith et al., 2020; Can et al., 2020) and incubated at 37° C. under 5% CO₂ for 72 hours. Cells were treated with concanavalin A (10 μg/ml) (Sigma Aldrich, USA) or cell stimulation cocktail (ThermoFisher Scientific, USA) as positive control and medium as negative control.

### Cytokine ELISA

In a preferred application of the invention; IL-4 and IFN-γ levels in stimulated splenocyte culture supernatants were determined by ELISA kits according to the manufacturer's protocol (ThermoFisher Scientific, USA). Briefly, 100 μl/well of splenocyte supernatants were added to each well of the plates and absorbance values were measured at 450 nm using a microplate reader (Bio-Tek EL×808, USA). Serial dilutions of mouse IL-4 and IFN-γ proteins provided by the kit were used to generate standard curves and calculate the cytokine level in cell culture supernatants. The detection limit for IL-4 and IFN-γ was 4 μg/ml and 15 μg/mL, respectively.

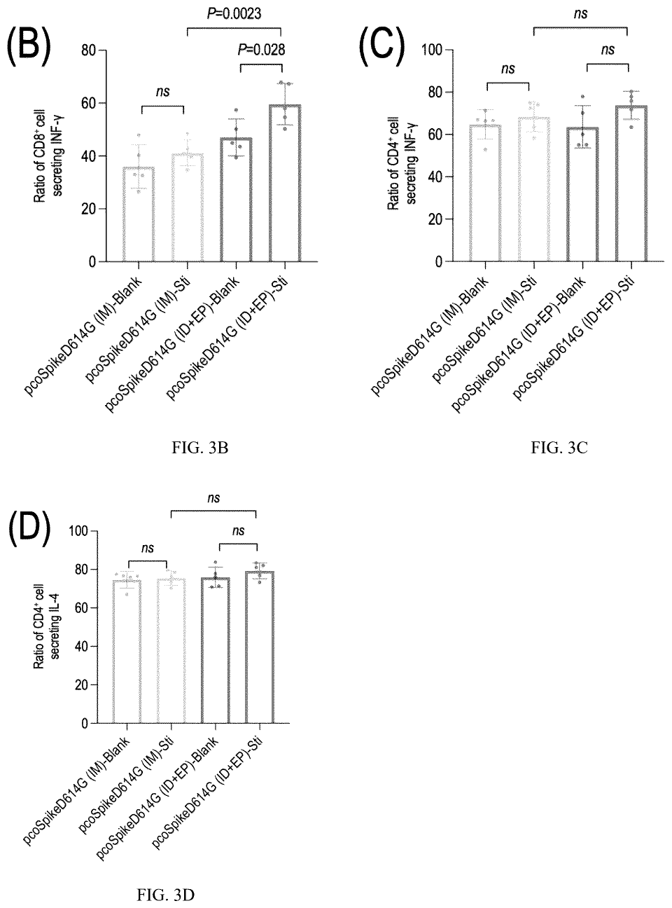

Extracellular cytokine levels calculated from supernatants of cultured splenocytes stimulated with the peptide pool for 72 hours showed that mice treated with pcoSpikeD614G via IM and ID+EP routes had mean IFN-γ levels of 1528.12 pg/ml and 1981.25 pg/ml, significantly higher than unstimulated cells (P=0.0029; P<0.0001). There was no significant difference between IFN-γ levels of mice treated with pcoSpikeD614G via IM and ID+EP routes. The mean IL-4 levels of mice treated with pcoSpikeD614G via IM and ID+EP routes were 3.87 pg/ml and 21.07 pg/ml, and the difference between unstimulated cells was not significant (FIG. 3A).

### Flow Cytometry

In a preferred application of the invention; Stimulated splenocytes to assess the percentage of IL-4 secreting CD4+ T cells as well as IFN-γ secreting CD4+ and CD8+ T cells, Alexa un 647 conjugated anti-CD3 (0.25 μg/well), FITC-conjugated anti-CD8 (0.5 μg/well), PerCP-cyanine 5.5-conjugated anti-CD4 (0.5 μg/well), PE-Cyanine 7-conjugated anti-IL-4 (0.2 μg/well) and PE-conjugated anti-IFN-γ (0.2 μg)/well) antibodies (ThermoFisher Scientific, USA) were used. An intracellular fixation and permeabilization buffer set (ThermoFisher Scientific, USA) was used for fixation and permeabilization of splenocytes. Stained cells were analyzed using a BD LSRFortessa™ cell analyzer and BD FACSDiva 8.0.1 software (BD Bioscience, USA).

To determine the proportions of IFN-γ secreting CD8+ and CD4+ cells and IL-4 secreting CD4+ cells, flow cytometry assays were performed with cultured splenocytes stimulated with the peptide pool for 72 hours. Accordingly, the proportion of IFN-γ secreting CD8+ cells increased in both groups of mice immunized with pcoSpikeD614G administered by IM and ID+EP routes. In the mouse group immunized with pcoSpikeD614G administered by IM route, the mean proportion of CD8+ cells secreting IFN-γ increased by 5.24%, but this increase was not statistically significant. In the mouse group immunized with pcoSpikeD614G administered via ID+EP, the mean proportion of CD8+ cells secreting IFN-γ was significantly increased by 12.51% (P=0.028). Furthermore, the proportion of IFN-γ secreting CD8+ cells was significantly higher in the mouse group immunized with pcoSpikeD614G administered via ID+EP than in the mouse group immunized with pcoSpikeD614G administered via IM (P<0.0023) (FIG. 3B). The proportion of CD4+ cells secreting IFN-γ was also increased in both groups of mice immunized with pcoSpikeD614G administered via IM and ID+EP routes. In mice immunized with pcoSpikeD614G administered via IM and ID+EP routes, the mean proportions of CD4+ cells secreting IFN-γ were increased by 3.78% and 10.19% compared to unstimulated cells (FIG. 3C). The proportion of IL-4 secreting CD4+ cells increased by 0.91% and 3.34% in both groups of mice immunized with pcoSpikeD614G administered via IM and ID+EP routes (FIG. 3D).

Challenge of K18-hACE2 Transgenic Mice with SARS-COV-2 Virus

Immunized K18-hACE2 transgenic mice were challenged with SARS-COV-2 virus (hCoV-19/Turkey/Pen07/2020, EPI_ISL_491476) isolated at Pendik Veterinary Control Institute. Two weeks after the third vaccination, mice were challenged with 105 TCID50 virus administered intranasally for 3 consecutive days at the Animal BSL-3 facility of TÜBiTAK MRC. Mice were checked for clinical symptoms and weighed daily, and general pathologic examination of organs was performed on day 15 of instillation. Lungs were scored to evaluate pneumonia (Ülbegi Polat et al. 2023a). Accordingly, healthy lungs without lesions were scored as 0, edematous and hyperemic lungs as 0.5-1, pneumonia lesions at different rates as 1.5-5 and dead mice as 5. Lungs were also collected for histopathologic examination and detection, and virus load was determined by RT-qPCR as described previously (Kayabolen et al., 2022; Ulbegi Polat et al., 2023b).

RT-qPCR targeting the nucleocapsid (NC) gene of SARS-COV-2 was performed as described previously (Ulbegi Polat et al., 2023b). For histopathological examination, lungs were treated with 10% buffered formalin for 48-72 h, dehydrated in a series of alcohol solutions of increasing concentration and embedded in paraffin wax. Sections were cut to a thickness of 5 μm and stained with hematoxylin and eosin for 5 minutes. Slides were visualized under a Zeiss Axio Vert A1 microscope (Zeiss, Germany) and zen software V2.6 (Zeiss, Germany). Semi-quantitative assessment was used in the study. Inflammation status in the lungs was compared with the whole lung and scored between 0-3. Accordingly, 0=No inflammation; 1=Normal morphology but mild erythrocyte and lymphocyte infiltration around the bronchioles; 2=Moderate erythrocyte and lymphocyte infiltration in the lung; 3=Disturbed morphology and intense erythrocyte and lymphocyte infiltration in the lung. Lungs of dead mice were not examined.

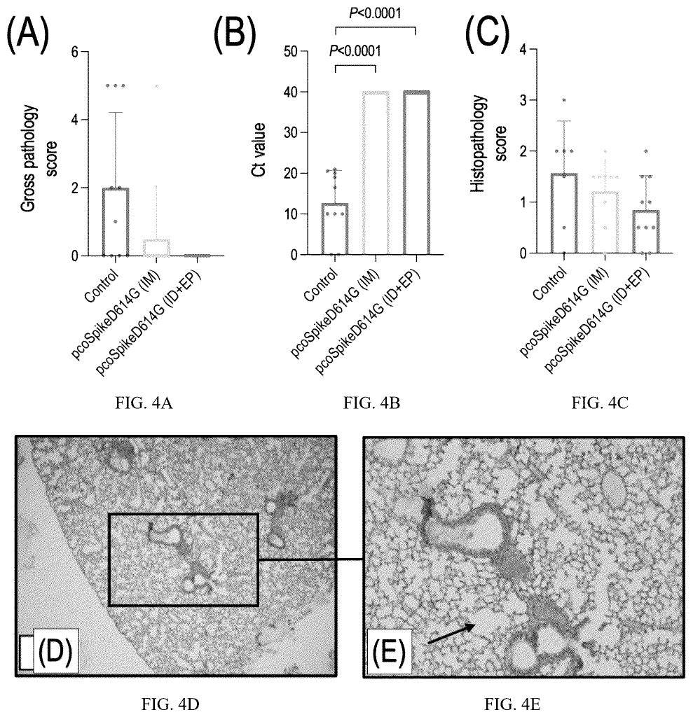

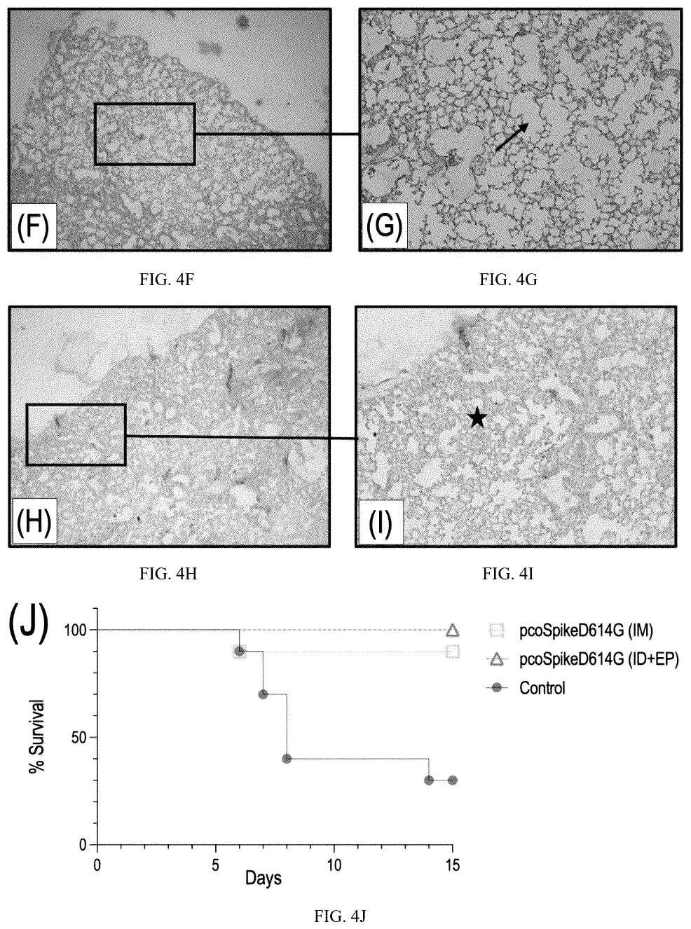

Two weeks after the third vaccination, K18-hACE2 transgenic mice were intranasally inoculated with 10⁵ TCID50 virus for 3 consecutive days. At 15 days after inoculation (not infected), the lungs of the mice were removed for gross pathological and histopathological examination, and RT-qPCR targeting the nucleocapsid (NC) gene of SARS-COV-2 was also performed to assess the virus load in the lungs of the mice. According to gross pathology scoring, the lungs of mice immunized with pcoSpikeD614G administered via ID+EP (n=10) and pcoSpikeD614G administered via IM (n=9) were intact, healthy and pink in color. In the control group, mice had moderate pneumonia in the lungs (FIG. 4A). In the mice that became ill and died before the end of the experiment, the signs of pneumonia indicating the course of the disease did not reach a level that could be seen with the naked eye. Visible pathological changes were observed in the lungs of some of the control group animals. In particular, the lungs were edematous, hyperemic and gray/brown discoloration was observed at the microscopic level, representing disruption of integrity. The mean gross pathological scores for mice immunized with pcoSpikeD614G administered by ID+EP and IM routes were 0 and 0.5, while the mean score of the control group was 2 (FIG. 4A). During RT-qPCR, the virus load in the lungs of mice was represented by Ct (crossing point threshold) values. Accordingly, the virus load in the lungs of control group mice (mean Ct: 12.7) was significantly higher than that of mice immunized with pcoSpikeD614G administered via IM (mean Ct: 1) (P=0.0004) and ID+EP (mean Ct: 0) (P<0.0001) routes (FIG. 4B). Histopathologic scoring of the lungs showed that the level of inflammation was 1.57, 1.2 and 0.85 in the control group and in pcoSpikeD614G administered via IM and ID+EP routes, respectively (FIG. 4C). Histopathological lung examination of control group mice showed lymphocyte infiltration areas in the interalveolar spaces (FIGS. 4H-4I), whereas mice immunized with pcoSpikeD614G administered via IM and ID+EP routes did not show any signs of inflammation in the alveoli (FIGS. 4D-4E and FIGS. 4F-4G). After intranasal instillation of 105 TCID50 viruses for 3 consecutive days, 30% of the control group, 90% of the mice immunized with pcoSpikeD614G administered by IM route and 100% of the mice immunized with pcoSpikeD614G administered by ID+EP route survived (FIG. 4J).

### Statistical Analysis

Normality of the data was examined and normal distribution was confirmed by column analysis using normality and lognormality of GraphPad Prism. Prism 10 software (GraphPad, USA) was used for statistical analyses including unpaired t-test, Mann-Whitney test and Kaplan-Meier survival analysis. Data were considered significant if P<0.05. Data in all parametric tests were expressed as mean±standard deviation (SD).

Data for all parametric tests are presented as mean±standard deviation (SD), while data for the nonparametric test (Microneutralization assay) are presented as geometric mean titer (GMT)±standard deviation (SD).

### INDUSTRIAL APPLICATION

The invention has the potential to be developed into products by biotechnology and pharmaceutical companies and used for the prevention of SARS-COV-2 infection.

What is claimed is:

1. An SARS-COV-2 recombinant Spike protein comprises the amino acid sequence containing a D614G mutation as defined by SEQ ID NO: 1, wherein the SARS-COV-2 recombinant Spike protein is used as a protective vaccine against SARS-COV-2 infection.

2. The SARS-COV-2 recombinant Spike protein of claim 1, wherein the SARS-COV-2 recombinant Spike protein is a fusion protein comprising an additional polypeptide, wherein the additional polypeptide comprises at least one of a signal peptide, a leader peptide sequence, and a detectable tag.

3. The SARS-COV-2 recombinant Spike protein of claim 2, wherein the signal peptide is selected from natural signal peptides of IgE.

4. The SARS-COV-2 recombinant Spike protein of claim 3, wherein the signal peptide is the amino acid sequence defined by SEQ ID NO: 2.

5. A nucleic acid molecule encoding the SARS-COV-2 recombinant Spike protein of claim 1, wherein the nucleic acid sequence is defined by SEQ ID NO: 3.

6. A nucleic acid molecule encoding the SARS-COV-2 recombinant Spike protein of claim 1, wherein the nucleic acid molecule comprises a nucleotide sequence encoding a signal peptide defined by SEQ ID NO: 4.

7. The nucleic acid molecule of claim 5, wherein the nucleic acid sequence is codon optimized or non-codon optimized for a codon preference of a host cell.

8. The nucleic acid molecule of claim 7, wherein the host cell is a human cell.

9. A vector comprising the nucleic acid molecule of claim 5, wherein the vector is a plasmid.

10. The vector of claim 9, wherein the vector expresses the SARS-COV-2 recombinant Spike protein containing the D614G mutation in a host.

11. The vector of claim 9, wherein the vector is for use in gene therapy.

12. A host cell comprising the nucleic acid molecule of claim 5 or a vector comprising the nucleic acid molecule.

13. The host cell of claim 12, wherein the host cell is a prokaryotic cell or a mammalian cell.

14. A method for expressing or producing the SARS-COV-2 recombinant Spike protein of claim 1, comprising using a nucleic acid molecule encoding the SARS-COV-2 recombinant Spike protein, a vector comprising the nucleic acid molecule, or a host cell comprising the nucleic acid molecule or the vector.

15. A pharmaceutical composition comprising the SARS-COV-2 recombinant Spike protein of claim 1 or a nucleic acid molecule encoding the SARS-COV-2 recombinant Spike protein or a vector comprising the nucleic acid molecule and a pharmaceutically acceptable carrier and/or excipient and/or adjuvant.

16. The pharmaceutical composition of claim 15, wherein the pharmaceutical composition is administered intradermally or intramuscularly.

17. The pharmaceutical composition of claim 15, wherein the pharmaceutical composition is a vaccine.

18. The pharmaceutical composition of claim 15, wherein the pharmaceutical composition is a protein vaccine comprising the SARS-COV-2 recombinant Spike protein.

19. The pharmaceutical composition of claim 15, wherein the pharmaceutical composition is a nucleic acid vaccine comprising the nucleic acid molecule or the vector.

20. The pharmaceutical composition of claim 19, wherein the nucleic acid vaccine is a DNA vaccine.
